(19) United States
(12) Patent Application Publication
(10) Pub. No.: US 2002/0155990 A1
(43) Pub. Date: Oct. 24, 2002

(54) Method of optimizing growth hormone replacement

(75) Inventor: Michael B. Foster, Phoenix, AZ (US)

Correspondence Address: Wood, Herron & Evans, L.L.P., 2700 Carew Tower, 441 Vine Street, Cincinnati, OH, 45202-2917, US

(73) Assignee: Renasci, Inc., d/b/a Renasci Anti-Aging Center

(21) Appl. No.: 09/939,962

(22) Filed: Aug. 27, 2001

Related U.S. Application Data
(63) Continuation-in-part of application No. 09/838,968, filed on Apr. 20, 2001.

(51) Int. Cl. G06F 19/00; G01N 33/48; G01N 33/50; A61K 38/27

(52) U.S. Cl. 514/2; 702/19

(57) ABSTRACT

A method to replenish human growth hormone (hGH) in a human adult. A initial daily dose of hGH is administered for three to four weeks, then an individualized maintenance dose is determined by determining the individual's response to serially increased doses of the initial dose. The maintenance dose is then administered, usually in a microsphere formulation so that reduced dosing frequency is possible. The method is useful to alleviate some of the effects of aging in mature adults.

### RELATED APPLICATIONS

This application is a continuation-in-part of U.S. patent application Ser. No. 09/838,968, filed on Apr. 20, 2001.

### FIELD OF THE INVENTION

The invention relates to a method to determine therapeutic regimens of human growth hormone administration in adults, particularly as an anti-aging therapy.

### BACKGROUND

The aging process in humans has physiological and psychological manifestations. In the musculoskeletal system, bone density, muscle mass, and lean body mass decrease. Fat body mass increases. Serum lipid levels change, for example, the ratio of “good” and “bad” cholesterol changes. Skin tone and elasticity decrease. Cerebral function decreases. Sexual function decreases.

It is known that administration of human growth hormone (hGH) can reduce, at least to some degree, the above-mentioned effects of aging. Human GH is a polypeptide that is naturally produced by the pituitary gland. Human GH drives the process of normal rapid growth during childhood and adolescence, regulating a variety of functions in virtually all cell types involved in the process. The effects of hGH are most easily observed as growth in muscle, bone, and skin.

Human GH has been used clinically since the 1970s to treat children with growth deficiencies. In the past, the only way to obtain hGH was to isolate it from cadavers, leading to safety concerns because of possible contamination and disease transmission. Now, hGH is synthesized in ultrapure form using recombinant techniques, and can be safely administered.

Besides treating growth deficiencies in children, hGH received approval by the Food and Drug Administration (FDA) in 1997 for use in the treatment of growth hormone deficiency states in adulthood, either as an isolated hormone deficiency or as part of a global pituitary deficiency profile. The approved indication requires that the adult recipient of hGH have either manifested the deficiency in childhood or adolescence, or have a specific, identifiable cause of a deficiency in pituitary function such as head trauma, surgery, irradiation, etc.

It is known that the level of hGH production declines with age, so that the amount of hGH in a 40-50 year old male is less than one-half of the level in an 18-25 year old male. As knowledge of the benefits of hGH replacement therapy become widespread, the use of hGH in adults continues to increase. However, treatment with higher doses of hGH (doses that are two to three times the mean doses reported in most of the literature regarding GH deficient patients) may produce troublesome side effects. Examples of such side effects include edema, joint and muscle pain, and entrapment defects as occurs in carpal tunnel syndrome. These effects have been reported in about one-third of participants in the small number of clinical trials employing doses of hGH that are much larger than the typical doses reported in the treatment of GH deficient adult patients. Furthermore, the FDA approved indication for hGH treatment in adults excludes this recognized age-related decline in GH secretion as qualifying under the definition of GH deficiency.

The dose and treatment regimen of hGH in adults, however, still remains problematic. No scientifically validated standards for induction and maintenance phases of therapy have been promulgated. The single method that has so far been established uses only a subtherapeutic dose of hGH. While this dose assures that recipients will avoid troublesome side effects, it does not attend to the individual needs and responses of recipients. Moreover, this method used hGH in a regimen that has no hGH enhancing effects; rather, it employs several low potency anabolic steroid hormones in pharmacological doses, which collectively have the effect of only mimicking some responses to hGH.

Thus, methods that currently exist for treating adults with hGH use either an ineffective dose of hGH, which is masked by the side effects of high doses of anabolic steroids, or produce an unacceptably high risk of troublesome side effects. While it has been suggested that side effects may be minimized by carefully monitoring the dose of GH and adjusting it to produce optimum levels of insulin like growth factor 1 (IGF-1) (Carter Clinics in Geriatric Medicine, Vol. 11, pp. 735-748, November 1995), such a method has not been reported. In addition, such methods lack sufficient attention to individual needs and responses of the recipients. Thus, a method to optimize hGH replacement therapy in adults is needed.

### SUMMARY OF THE INVENTION

The inventive method provides for a composition that is administered to an adult in order to provide anti-aging effects. The method involves adminstering a composition of human growth hormone (hGH) that lacks other hormones or other bioactive compounds. The method employs an inductive dose of hGH, and considers the individual's own response to daily doses of hGH to determine the desired maintenance dose unique for that individual.

No other known methods provide for individualized dosing of a composition that contains only hGH as the active component. Furthermore, in contrast to methods which require daily or even more frequent dosing, the inventive method permits the maintenance dose to be administered as infrequently as on a monthly basis by dosing hGH in a time-released formulation, such as a microsphere. This provides convenience to the individual and removes the unpleasantness of frequent injections.

The inventive method also provides a method and system for optimizing human growth hormone (hGH) replacement in a patient. The system uses a specially programmed computer that communicates between a specialist physician in hGH replacement, who directs and monitors patient screening and treatment, and a non-specialist health professional, usually a physician or nurse, who doses and monitors the patient and communicates the information to the specialist for evaluation. This method is particularly beneficial where a patient otherwise could not receive treatment because he or she was not in the geographical vicinity of the specialist. In the method, patient data, including a level of insulin like growth factor 1 (IGF-1), are analyzed by the program to determine if the patient is a candidate for hGH therapy. If the patient is a candidate, the program calculates the initial dose for that patient, and the dose is verified by the specialist. Upon verification, the non-specialist health professional adminsters the pre-determined dose, entering the information into the program. In one embodiment, the non-specialist professional has at his or her disposal an array of vials containing pre-dose hGH, with each vial containing the dose information in both human and computer readable formats, so that the non-specialist professional simply scans the vial to enter the dose information for the particular patient into the program. The specialist monitors patient dosing and clinical data, such as IGF-1 levels, throughout treatment, including the initial dose and the maintenance dose of hGH, and communicates the desired treatment regimen for that patient and other information via the computer to the specialist.

The invention will be further appreciated in light of the following drawings, detailed description, and examples.

### BRIEF DESCRIPTION OF THE DRAWINGS

FIG. 1 is a schematic illustration of the system components.

FIG. 2 is a schematic general illustration of the method.

FIG. 3 is a schematic illustration of one embodiment of the program.

### DETAILED DESCRIPTION

A method is disclosed to replenish the age-related decline in human growth hormone (hGH) in adults by administering an individualized dosing regime of hGH in the absence of any other bioactive compounds. In the method, the individual initially receives incrementally increasing doses of hGH (inductive dose), while undergoing physiological and/or sociological assessment to determine the effect of hGH. Bases on these outcomes, a maintenance dose to achieve the desired hGH replenishment for the individual is then determined. Thereafter, the individual receives this maintenance dose of hGH, either on a daily or monthly basis, depending upon his or her preference. Outcomes of this method of individualized hGH therapy include increased bone density, muscle and lean body mass, decreased fat body mass, improvement in serum lipid levels, for example, the ratio of “good” and “bad” cholesterol, improvement in skin tone and elasticity, improved cerebral function, improved sexual function, and an improved general sense of well being.

The individual undergoing therapy is an adult, either male or female and typically at least 40 years old, who is in a general state of good health. Good health is assessed by a preliminary examination, including a complete medical history with a complete list of all medications taken regularly. A screening test for prostate specific antigen (PSA) is performed on male patients. A physical examination is also performed, including hematological and chemical panels to determine overall health, biological age and current levels of various hormones such as hGH, testosterone, estrogen in women, and insulin like growth factor 1 (IGF-1). Hormone levels are charted and compared to normal baseline levels for biological age to determine the extent of their depletion, and as a baseline from which to assess therapeutic outcomes. Adults treated with thyroid hormone, testosterone, and estrogen are amenable to treatment according to the inventive method, and the parameters in their preliminary evaluation will be valid as long as the dose of the medication is stable. If clinical assessment indicates levels of testosterone or estrogen that are too high, or levels of thyroid hormone that are either too low or too high, the dose must be adjusted and blood levels of the offending medication must be permitted to reach a new equilibrium before proceeding with the inventive method.

An individual-specific treatment with hGH is then initiated. A stable liquid hGH formulation is used to gradually replenish hGH. This allows an optimum physiological replacement dose of hGH to be adminstered to the individual with a risk of side effects to be within an acceptable range. In contrast to known methods, the inventive method does not administer adjunctive agents such as other hormones or bioactive compounds such as melatonin or vitamin B12.

The hGH hormone administered is a commercially available synthetic peptide (examples of which include but are not limited to Nutropin®, Genetech, San Francisco, Calif.; Genotropin®, Pharmacia, Peapack, N.J.; Humatrope®, Eli Lilly, Indianapolis, Ind.; Norditropin®, Novo Nordisk, Princeton, N.J.; Serostim®, Serono, Norwell, Mass.), produced by recombinant molecular biology techniques. Administration of hGH is by parenteral means. In one embodiment, hGH is administered by subcutaneous injection. Injection may be in the arm, leg, stomach, buttock, or hip.

A daily injection of hGH is adminstered in an initial phase of therapy. A stable solution of hGH (e.g., Nutropin AQ®, Genentech, San Francisco, Calif., and other products) is administered at a dose of about 2 μg/kg/day in males and about 4 μg/kg/day in females. Daily injections of this initial dose are continued for about three to four weeks.

The initial dose of hGH is then titrated to an adjusted, or maintenance, dose. The maintenance dose is that which produces the desired level of hGH replenishment for that individual. Maintenance doses are typically about 10-14 μg/kg/day for males, and 14-20 μg/kg/day for females, and are determined by physical response and attainment of desired levels of IGF-1. Since IGF-1 is produced in response to growth hormone, the level of IGF-1 serves as a mediator of the anabolic effects of hGH therapy in adults, as well as statural response in children. Measurement of circulating levels of IGF-1 provides an accurate index of an integrated measure of GH level and effect. Furthermore, whereas GH levels are very volatile and difficult to interpret clinically, IGF-1 levels are exceedingly stable and can be assayed in blood samples that are drawn at any time of day.

The maintenance dose of hGH is determined by evaluating the individual's response to serially increased doses of hGH, usually over one to two months. The dose is adjusted at about two to four week intervals, and in a range equal to that of the initial dose. For example, a male receiving an initial dose of 2 μg/kg/day would receive a serially increased dose of 4 μg/kg/day for two to four weeks, then a dose of 6 μg/kg/day for two to four weeks, then a dose of 8 μg/kg/day for two to four weeks, etc., until the maintenance dose is achieved. A female receiving an initial dose of 4 μg/kg/day would receive a serially increased dose of 8 μg/kg/day for two to four weeks, then a dose of 12 μg/kg/day for two to four weeks, etc., until the maintenance dose is achieved.

Dosing may be accomplished using a unit dose system (Pharmacia) or a multidose vial. In a multidose system, a cartridge separately contains lyophilized hGH and diluent. When mixed, a stock hGH solution is obtained, the proper dose selected by the volume of solution that is administered.

Once the maintenance dose is achieved, a monthly dose of hGH is administered. This may be done by administering hGH in a time-released formulation, such as a microsphere formulation (e.g., Nutropin Depot®, Genentech, San Francisco, Calif., and other products). To calculate the monthly dose, individualized bioavailability data are determined, since the microsphere formulation has 10-20% less bioavailability than daily dose formulations. While monthly administration regimens are available, that is, only one injection per month, and in fact may be preferred because of scheduling or other demands, a daily regimen is equally effective for those recipients desiring daily administration for psychological or other reasons.

The invention also encompasses a method whereby candidates for therapy with hGH are evaluated, selected, treated, and monitored during treatment. Patients are under the care of a specialist in hGH replacement therapy, while remaining in their own geographical locale and physically attended to by their own physician or other health professional. Normally, a general practitioner or other health professional routinely caring for the patient desiring hGH therapy would not have the background, training, expertise or information necessary to administer, monitor, alter, and assess outcomes of hGH therapy. Patients not able to be seen by a specialist would therefore be denied the opportunity to be evaluated for, and possibly receive, hGH therapy.

The inventive method addresses this problem and provides for remote individualized evaluation, selection, treatment, monitoring and adjustment of therapy under the guidance and monitoring of a specialist in hGH replacement, but with the hands-on patient care performed by physicians or other health professionals who lack the specific training or expertise in hGH replacement. Therefore, two health professionals are involved, with the specialist professional having specialized education, training, and/or experience in this area, but at a location remote from the patient, and the non-specialist professional having education, training, and experience in clinical medicine to be able to assess and treat the patient and also to communicate with the specialist concerning the patient's medical condition. Specific protocols and procedures established by the specialist are followed and reported by the non-specialist, with communication between the two professionals at least via the computer program. The method thus benefits patients desiring hGH replacement who otherwise would not be able to take advantage of and benefit from its effects. The method additionally optimizes and standardizes the selection of candidates who will likely benefit from hGH replacement, the screening of these candidates for the dose of hGH to be administered, and the parameters by which to evaluate hGH replacement in these candidates.

Potential candidates are typically at least 40 years old and are in a general state of good health, as determined according to the parameters previously described. The non-specialist professional obtains a patient sample such as blood, serum, or interstitial fluid for determination of insulin like growth factor 1 (IGF-1) levels, since IGF-1 is a marker for growth hormone. IGF-1 levels can be determined by immunoassay (e.g., Quest Diagnostics/Nichols Institute, San Juan Capistrano, Calif.). A patient having a lower than normal level of IGF-1 is a candidate for hGH therapy. However, as will be appreciated by one skilled in the art, a “normal” IGF-1 value varies with gender, age, and other parameters, and also varies among textbook or other reference sources. Therefore a relative, versus absolute, variation from normal is used to determine a low IGF-1 level. As general guidelines, a specialist could consider an IGF-1 level low if it is below a reference reported mean IGF-1 value minus one standard deviation, or if it is at least about 5% below a reference reported normal value. In one embodiment, patients having an IGF-1 level <330 ng/dl are candidates for hGH treatment. In another embodiment, patients having an IGF-1 level <303 ng/dl are candidates for hGH treatment.

If the IGF-1 level is not low, defined as previously described, the result is charted in the patient's file, but therapy with hGH may not be initiated. If the IGF-1 level is low, the result is charted in the patient's file and the patient is identified as a candidate for hGH therapy, if other medical parameters are within normal limits and there are no other confounding factors. The IGF-1 level is one factor of a number of factors the specialist considers in evaluating candidacy for hGH therapy; for example, even if the level of IGF-1 is not low, a patient may be a candidate for hGH therapy if his or her level of testosterone is low. Other growth hormone-dependent parameters may also be evaluated, such as insulin like growth factor binding proteins 1-4 (IGFBP-1, IGFBP-2, IGFBP-3, and IGFBP-4, particularly IGFBP-3 and its acid labile subunit (ALS).

While levels of IGF-1 and testosterone are independent indicators for their respective therapies, the level of IGF-1 is influenced by the level of testosterone. A “normal” testosterone level may vary, as previously described for IGF-1, so evaluation of a relative, versus absolute, testosterone level is preferred. For example, reference reported testosterone levels are 572±135 ng/dl in an adult male, and 37±10 ng/dl in an adult female (Fundamentals of Clinical Chemistry, Norbert W. Tietz, Ed., W. B. Saunders Co., Philadelphia 1987). Thus, in an adult male a low normal testosterone level is about 437 ng/dl (572 ng/ml-135 ng/ml); an adult male having a testosterone level at least 10% lower than the low normal level would be considered a candidate for hGH therapy. Similarly, in an adult female a low normal testosterone level is about 27 ng/ml (37 ng/ml-10 ng/ml); an adult female having a testosterone level at least 30% lower than the low normal level would be considered a candidate for hGH therapy.

Because of the role of hGH as a hormone, as well as other reasons, it is beneficial to monitor levels of other hormones in an individual undergoing hGH replacement therapy. For example, the specialist physician may query the patient and/or general physician about the patient's level of thyroid hormones, and may test for thyroid function (e.g., levels of T₃, T₄, and thyroid stimulating hormone (TSH)).

If the patient is a candidate for hGH replacement therapy, the specialist determines the initial dose of hGH for the patient. The patient dose is calculated using a program that includes parameters such as gender, age, and weight, the results from hematological and chemical panels, hormone levels such as IGF-1 testosterone, estrogen, T₃, T₄, TSH, etc., and other parameters to assess overall health (e.g., medical history, frequency of tobacco and/or alcohol use, blood pressure, use of medications (both prescription and over the counter), cardiovascular fitness, etc.). An initial dose of hGH is then determined for the individual. The initial hGH dose is also used for future reference, as it is the maximum value by which incremental increases in dose can be adjusted; that is, no increased dosage is administered to the patient in which the increment is greater than the initial hGH dose. The calculated initial dose of hGH is then charted in the patient's file and is stored in the patient data program.

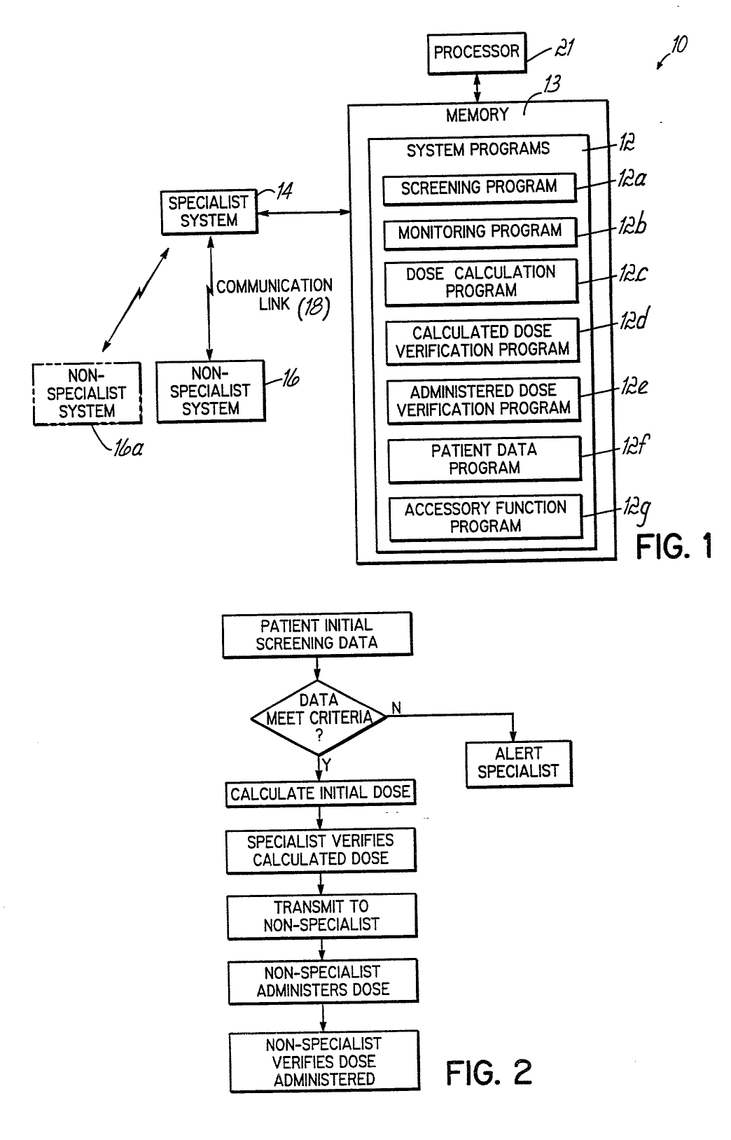

The above-described process is implemented using a computer based system 10 such as that shown in FIG. 1. To perform the desired calculations and monitoring, system 10 is typically under the control of system programs 12 that are resident in memory 13. The system 10 also contains a processor 21 that uses the system programs 12 to perform the necessary functions. It should be appreciated that the system programs 12 may be stored in a specialist system 14 (a component accessible to the specialist physician), on a separate network (not shown), or on mass storage devices (not shown) prior to start-up. In addition, it may have various components that are resident at different times in any of memory 13, network, or mass storage, or within registers and/or caches in the processor 21 (e.g., during execution thereof). It should also be appreciated that other software environments may be utilized in the alternative.

Besides a component that is accessible to the specialist system 14, the system 10 has a non-specialist system 16 (a component accessible to the non-specialist health professional). Only the specialist, however, has access capability to the dose calculation programs 12c. This type of controlled access prevents alteration of parameters regarding patient dosing by anyone other than the specialist. The specialist accesses the system programs 12, using means such as password entry, magnetic card, fingerprint or voiceprint recognition, corneal scans, etc. for authentication, as known to one skilled in the art. It is also anticipated that, in select cases, the non-specialist system 16 of system 10 may be accessed by a patient who communicates with the specialist and self-administers the dose determined by the specialist. Each user's access to the system is based on authentication of that user.

The specialist system 14 at one location interfaces with the non-specialist system at a geographically remote location via a communications link 18. Use of the communications link 18 may be by any type of input/output device such as a keyboard, mouse, voice command, telephone, modem, memory card, etc., as known to one skilled in the art.

The system programs 12 are composed of a number of individual programs. These programs may include, for example, a screening program 12a, a monitoring program 12b, a dose calculation program 12c, a calculated dose verification program 12d, an administered dose verification program 12e, a patient data program 12f, and an accessory function program 12g. The screening program determines whether the patient is a candidate for hGH therapy, using the criteria previously described. The monitoring program 12b monitors at least a patient's level of IGF-1, and may also monitor a patient's level of testosterone, other hormones, and any other patient-specific parameters the specialist and/or non-specialist desires to monitor before and/or during treatment. The dose calculation program 12c calculates the dose of hGH to be administered, depending upon general and patient-specific variables as previously described. The calculated dose verification program 12d allows the specialist to verify the calculated hGH dose before this information is transmitted to the non-specialist system 16. The administered dose verification program 12e allows the non-specialist to verify that the specific dose was administered. The patient data program 12f maintains patient-specific information. The accessory function program 12g allows for queries and responses between the specialist and non-specialist, provides a signaling function to alert the specialist or non-specialist, etc. Other programs may also be included.

The specialist system 14 is monitored and supported by physicians and/or other health care professionals who have the required training and expertise in hGH replacement therapy. One or more non-specialist systems 16a, 16b, etc. are used by non-specialists, and hence will typically be located in the hospitals, clinics or offices of general physicians or health professionals who physically interact with the patients undergoing treatment, but who have less or no training or expertise in hGH dosing and therapy. The specialist system 14 may contain the system programs 12, as previously stated. Alternatively, the system programs 12 may be at a third site and operated with an independent processor 21.

FIG. 2 is a flow chart generally illustrating the inventive method. Typically, the specialist has the patient present to a non-specialist professional, who performs the initial screening tests to determine if the patient is a candidate for hGH anti-aging therapy. These data are entered into the system 10, and the screening program 12a determines whether the data meet the criteria required of a candidate for hGH therapy, as previously described (i.e., low levels of IGF-1 and/or testosterone, hematology and chemistry panels within acceptable limits, over 40 years old, etc.). If the data do not meet these criteria, the specialist is alerted and may request further information, consultation, or testing.

If the data do meet these criteria, the dose calculation program 12c calculates the initial hGH dose for the patient. This calculation is based upon both general factors such as gender, age, and weight, and specific factors. As one example, a specialist determining an hGH dose for a patient having a markedly low initial IGF-1 level may initiate therapy with a lower than usual hGH dose, as this patient will be more sensitive than usual to hGH therapy. As another example, a specialist determining an hGH dose for a female patient on hormone replacement therapy may initiate therapy with a higher than usual hGH dose, as this will compensate for the known suppressor effect of estrogen on hGH. As yet another example, a specialist determining an hGH dose for a patient receiving combined hGH and testosterone therapy may initiate hGH therapy with a lower than usual hGH dose to better evaluate the effects of the combined therapy. As still another example, a specialist determining an hGH dose for a patient with an entrapment syndrome such as carpal tunnel syndrome would be cognizant of water retention experienced by these patients in setting the initial dose.

The calculated dose is transmitted to the specialist for verification. The specialist can then verify the dose, or query the patient and/or the non-specialist. Using the verification program for a calculated dose 12d, the specialist then instructs the non-specialist to either administer or not administer this dose. Once patient criteria and dose information are verified by the specialist, the initial dose to be administered is then transmitted to the non-specialist professional who will administer the hGH dose. Other information from the specialist may also be transmitted. As one example, the specialist may advise or require monitoring a patient with an entrapment syndrome for peripheral edema. As another example, the specialist may advise or require more frequent and/or detailed monitoring of a patient receiving both hGH and testosterone therapy for an augmented response. As yet another example, the specialist may advise or require more frequent or more detailed monitoring of blood pressure and/or blood lipids in a patient receiving testosterone therapy, due to the known hypertensive and lipid-altering effects of testosterone, as well as its effect to increase body fat as well as lean body mass. The specialist may also provide reminders of the required testing interval, etc. to the non-specialist. Notification may be accomplished by any sort of prompt, visual and/or auditory, using the computer system 10. The non-specialist can then, or at any time, query the specialist physician about the dose, and whether or not a change in the dose is possible or desirable. Likewise, the specialist can then, or at any time, query the non-specialist health professional about the patient. If the dose is administered, the non-specialist then verifies this using the verification program for an administered dose 12e.

To administer hGH, the non-specialist prepares the initial dose. In one embodiment, this non-specialist professional has access to a stock of sterile, sealed vials containing various hGH doses and labeled in both human readable form and computer readable form (such as with a bar code). In use, the appropriate vial is selected and entered into the program; for example, a bar code affixed to the vial containing this information is scanned using a hand-held scanner or other scanning apparatus to enter the encoded information into the calculated dose veritifcation program 12d. The method also allows the specialist another opportunity to verify the correct hGH dose; the program may be set up to require a second verification from the specialist or a specialist staff member before dosing to minimize error. The non-specialist then prepares the patient (e.g., disinfects the injection site), withdraws the correct dose (which may be the entire contents of the vial), and injects the patient with hGH. The program may be set up to require verification that the dose contained in the vial was administered, and allow any comments or questions to be entered for response by the specialist. For example, in transitioning a patient receiving daily hGH therapy to a depot formulation, the specialist may explain to the non-specialist that an interval of two or more days should be provided between doses.

In one embodiment, the monitoring program 12b can be accessed at any time, by the specialist, non-specialist, and/or patient to determine the patient's level of, for example, IGF-1. Similarly, the patient data program 12e can be accessed at any time to review patient data. However, as previously described, only the specialist has access to the dose calculation program 12c.

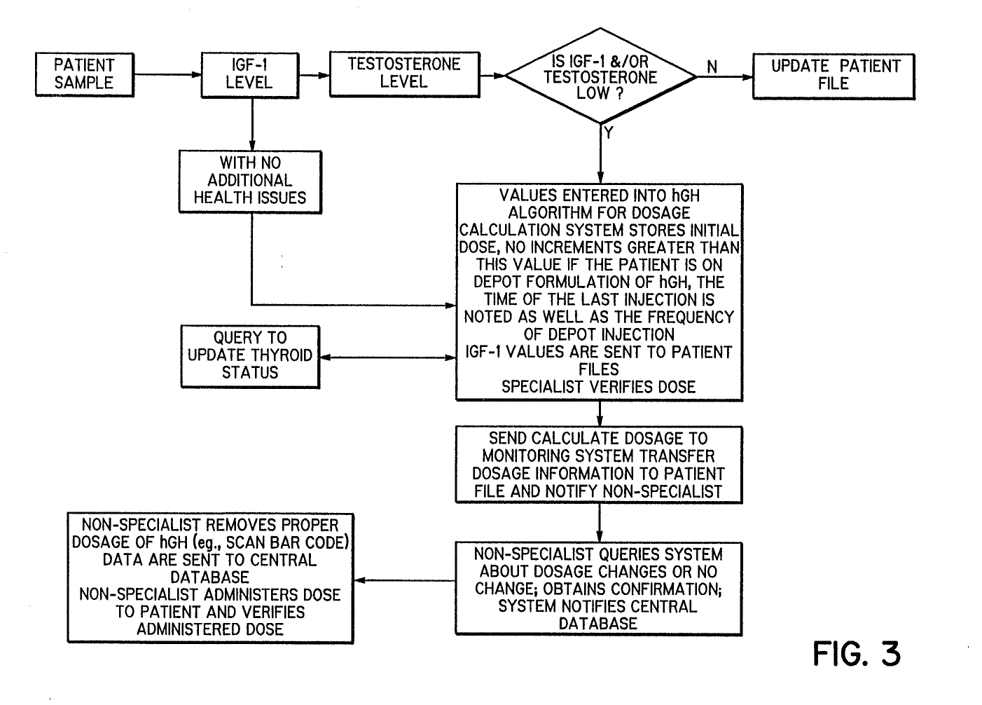

FIG. 3 shows a particular embodiment of the program for the inventive method, incorporating additional parameters. Either the specialist or the non-specialist analyzes the patient's sample for the level of IGF-1, and the results are analyzed by the program. If a new patient does not have a low IGF-1 level, as previously defined, the patient's testosterone level is obtained. In some cases, the patient's testosterone level is obtained regardless of the IGF-1level. If both IGF-1 and testosterone levels are at or above a defined threshold, these results are entered in the patient's file, but the patient is likely not a candidate for hGH therapy. If a new patient has a low IGF-1 level, as previously defined, the program analyzes other patient data to ensure there are no health-related reasons why the patient should not become a candidate for hGH therapy. Both male and female patients are questioned about thyroid problems, since normal thyroid function is needed for a normal response to hGH therapy.

The above data are entered into the program for calculating the initial dose of hGH. The calculated hGH dose is entered and incorporated into the program so that no increments greater than this value are administered to that specific patient. For a patient on a maintenance dose of hGH, and/or a patient receiving hGH as a depot formulation, the time of the previous injection and the frequency of the injection regimen is entered in the program. The depot formulation allows adjustment of both the amount of hGH administered as well as the interval between injections, thus it provides another alternative dosing parameter.

The calculated dose of hGH is entered into the program and transferred to the specialist. Upon verification of the dose by the specialist, the non-specialist is notified to verify the dose, and may also query the specialist regarding the dose. Upon satisfactory confirmation of the dose and any other issues, the specialist authorizes administration of hGH therapy.

The non-specialist professional selects and prepares the proper dose of hGH to be administered and enters this dose into the program. In one embodiment, this is accomplished using bar coded vials and scanning the information into the program, as previously described. The specialist may verify the dose, if desired. The dose is then administered to the patient.

Outcome assessment of therapy is based on one or more combinations of several objective and/or subjective parameters. In one embodiment, the program analyzes patient data and evaluates efficacy of the therapy. Objective outcome parameters include physical assessment such as total body weight, standing height, body composition as measured by electrical impedance or another type of measurement, tolerance to exercise, biochemical assessment such as measurement of blood levels of IGF-1 and other growth hormone-dependent parameters such as insulin like growth factor binding proteins 1-4 (IGFBP-1, IGFBP-2, IGFBP-3, and IGFBP-4, particularly IGFBP-3 and its acid labile subunit (ALS)), and psychological assessment such as memory tests. The measurement of each of these parameters is known to one skilled in the art. Subjective outcome parameters include responses to questionnaires concerning, for example, improvement in sexual function and a general sense of well being.

The following examples illustrate the inventive method.

### EXAMPLE 1

The subject was a 78 year old male retired professional. Throughout his career he had been a leader in his community and had been active in a variety of pursuits, ranging from daily exercise to an avid practice of several creative arts. As he progressed into his sixties, he found that he was experiencing an unacceptable decline in his physical capabilities. His capacity for exercise began to diminish and, because of his meticulous attention to his fitness, he was aware of progressive deterioration of his lean body mass. He also began to experience several orthopedic problems, including a change in his posture, which were very distressing to him. He was medically sophisticated, and so he turned to the remedies which were available to him, but he experienced frustratingly meager responses. Finally, he began to experience a decline in his creativity, which increased his sense of urgency in seeking an effective response to the ravages of aging.

After lengthy discussion of the possible alternatives, he elected treatment with an agent employing both testosterone and dihydroxyepiandosterone (DHEA), purported to stimulate native growth hormone secretion. The agent was completely ineffective in producing the desired response of a renewal of his physical capacities. The agent also did not produce a biochemical response in the form of restoration of his index of growth hormone activity to a level commensurate with robust good health for an adult. At this point in his history, and without achieving a satisfactory response, the subject began administration of human growth hormone using the inventive method.

A baseline IGF-1 level of 119 ng/ml confirmed that his level of GH secretion was low. Daily subcutaneous hGH therapy was begun at a dose of 150 μg/day (about 2.5 μg/kg/day). During the induction of hGH therapy, his self-prescribed daily oral supplement of dehydroepiandrosterone (DHEA) was eliminated from his routine.

After three weeks of therapy, in the absence of side effects and with his IGF-1 level elevated to 184 ng/ml, his hGH dose was increased to 300 μg/day (about 5 μg/kg/day). After an additional three weeks of therapy, his IGF-1 level rose to 237 ng/ml. Still in the absence of side effects, his daily hGH dose was increased to 450 μg/day (about 7.5 μg/kg/day). After a third three week interval, there were still no adverse effects and his IGF-1 level rose to 270 ng/ml. His daily hGH dose was increased to 600 μg/day (about 10 μg/kg/day). After a fourth three week interval, there were still no adverse effects and his IGF-1 level rose to 305 ng/ml. His IGF-1 level was stable above 300 ng/ml on a daily dose of 600 μg/day.

Once the appropriate therapeutic dose of human growth hormone was identified and administered, continued hGH therapy produced astounding physical changes. The subject's exercise capacity was the first parameter to undergo a noticeable change. Both endurance in aerobic exercise and strength in resistance exercise showed significant improvement. Posture was the next parameter to show a credible response. The change was noticeable even to casual observation by friends and associates who encountered the subject in his day-to-day life. Finally, restoration of lean body mass was exhibited in facial appearance and in measurement of chest, abdomen and upper arm circumference. The unsolicited response from observers who were unaware of the therapy was that he appeared more robust, fifter and, in a word, younger.

An unanticipated result of the therapy was that the subject began to experience an undoubted increase in his creative productivity. This did not appear to be solely the result of an increase in his physical capacity for work. What the subject described as his interior life was more vibrant. Ideas came in torrents as they had during his younger years, and he felt the restoration of his zeal to create. Simultaneously, he experienced a reawakening of his sense of sexual competence. The subject has regained the scope of interests and activities that characterized his middle years. Having known him over a period of a decade and a half, the word that comes to mind is “rejuvenation”.

### EXAMPLE 2

The subject was a male in his late 50's who had experienced significant concomitant fractures in both the tibia and fibula. In spite of intensive conventional therapy, the injury had failed to heal completely. An evaluation showed a baseline IGF-1 level of 74 ng/ml, revealing clear evidence of his lack of the beneficial influence of growth hormone. Therapy with hGH was begun in an attempt to enhance his healing.

Since his IGF-1 level was quite low, therapy with hGH was begun at a very low dose. The initial hGH dose was 100 μg/day, calculated on the basis of about 1.2 μg/kg/day). This produced an IGF-1 level of 91 ng/ml after a three week interval. The second hGH dose was 200 μg/day. After an additional three weeks of therapy, his IGF-1 level rose to 126 ng/ml. Still in the absence of side effects, his daily hGH dose was increased to 400 μg/day, with subsequent doses adjusted at three week intervals in increments equal to his second dose (that is, in increments of 200 μg/kg) because he remained free of any side effects through the first two three-week intervals. After a hGH dose interval of 400 μg/day, his IGF-1 level rose to 235 ng/ml. After a hGH dose interval of 600 μg/day, his IGF-1 level rose to 294 ng/ml. After a dose interval of 800 μg/day, his IGF-1 level rose to 321 ng/ml. He experienced no adverse side effects and required no ancillary hormone replacement therapy.

Within a few weeks of achieving an IGF-1 level above 300 ng/ml, a measurable improvement in the healing of his fractures was apparent. The therapy was continued until the injury had healed completely. During the period of initial therapy, the subject reported the restoration of a joie de vivre that had been lacking for a period of years. His physical therapy became enjoyable and, with the healing of his injury, he embarked upon a much more active lifestyle.

With the complete resolution of his original injury, the subject has elected to continue his therapy with hGH because of the improvement in his quality of life. He has abandoned his sedentary habits and is exploring a variety of new physical activities. He looks and feels more youthful, and the people in his life who are unaware of his therapy are surprised at the change that has taken place in his approach to living. He has brought innovations into his already successful business and he is finding new outlets for his creative energies. With complete healing of his initial injury, the option of discontinuing therapy has come under discussion, but he stated that he has no intention of relinquishing his hold on an enhanced life.

### EXAMPLE 3

The subject was a woman in her early 50's who had experienced problems with infertility, but who considered herself otherwise healthy. She had been plagued by a progressive decline in her endurance and in her enthusiasm for the normal range of activities in her life. She inquired about the possibility that a low level of growth hormone secretion might be contributing to the problems that she had been experiencing. She proved to have an IGF-1 level of 176 ng/ml, indicating a low level of growth hormone secretion. In light of that finding, she was ready to consider herself a candidate for hGH therapy.

Therapy with hGH was initiated at a dose of 200 μg/day (about 4 μg/kg/day). The hGH dose was raised in increments of 200 μg/day at four-week intervals to a maintenance hGH dose of 800 μg/day (about 15 μg/kg/day). These hGH doses produced IGF-1 levels as follows: an IGF-1 level of 194 ng/ml with a hGH dose of 200 μg/day, an IGF-1 level of 208 ng/ml with a hGH dose of 400 μg/day, an IGF-1 level of 243 ng/ml with a hGH dose of 600 μg/day, and an IGF-1 level of 297 ng/ml with a hGH dose of 800 μg/day. Her IGF-1 level was stable in the range of 300 ng/ml on a daily dose of 800 μg/day.

With her IGF-1 levels in the normal range, she began to experience a return to her former level of physical activity and a resurgence in enthusiasm for the other interests that had comprised her active and productive life. The subject had already experienced the menopause, and she was taking appropriate hormone replacement therapy. The introduction of hGH therapy did not require altering her other hormone replacement therapy in any way, but did serve to enhance her sense of well being which had been only partially restored by the traditional therapy.

The subject is now participating in daily aerobic exercise, including serving as an instructor in several classes per week. She has experienced an awakening of new enthusiasm for her career, and is making plans to expand the scope of her professional activities. She is monitoring her body composition and, though her weight has actually remained unchanged, she is leaner and stronger than she has been at any point in her life after adolescence.

It should be understood that the embodiments of the present invention shown and described in the specification are only preferred embodiments of the inventor who is skilled in the art and thus are not limiting in any way. Therefore various changes, modifications or alterations to these embodiments may be made or resorted to without departing from the spirit of the invention and the scope of the following claims.

What is claimed is:

1. A method of optimizing human growth hormone (hGH) replacement therapy in a patient comprising
providing patient data to a specially programmed computer having communication with a specialist in hGH replacement therapy located remote from said patient,
receiving information from said specialist regarding an individualized dose of hGH,
administering the individualized dose of hGH to said patient.

2. The method of claim 1 wherein said patient data includes data selected from the group consisting of an insulin like growth factor 1 (IGF-1) level, a testosterone level, a thyroid hormone level, and combinations thereof.

3. The method of claim 1 wherein said dose of hGH is provided in a container having dose information contained in a computer readable code, and said code is scanned into said programmed computer.

4. A method of determining whether a patient is a candidate for anti-aging therapy with human growth hormone (hGH) comprising
providing patient data selected from the group consisting of age, gender, hematology profile results, chemistry profile results, insulin like growth factor-1 (IGF-1) level, testosterone level, a thyroid hormone level, and combinations thereof to a specialist in hGH replacement therapy using a specially programmed computer,
thereafter following a directive by said specialist for treating said patient with hGH if said IGF-1 level is lower than a normal IGF-1 level or said testosterone level is lower than a normal testosterone level.

5. The method of claim 4 further comprising
providing said level of IGF-1 from a monitoring site where the patient is located to a specialist site where a hGH specialist is located,
determining said dose of hGH at said specialist site,
communicating said dose from the specialist site to the monitoring site, and
administering said determined dose at said monitoring site.

6. The method of claim 4 wherein said specialist monitors said patient administered with said hGH dose.

7. The method of claim 6 wherein said administered hGH dose is an initial dose.

8. The method of claim 6 wherein said administered hGH dose is a maintenance dose.

9. A method for monitoring a patient receiving human growth hormone (hGH) as an anti-aging therapy by a specialist in said therapy at a location remote from said patient comprising
evaluating patient medical data entered into a specially programmed computer communicating between said specialist and an on-site health professional to verify that said patient is a candidate for hGH therapy,
directing a dose of hGH to be administered to said patient and
monitoring said patient for responsiveness to said administered hGH dose.

10. The method of claim 9 further comprising a health professional at a location on-site of said patient entering said medical data.

11. The method of claim 9 further comprising a health professional at a location on-site of said patient administering said dose of hGH.

12. The method of claim 9 further comprising a health professional at a location on-site of said patient querying said specialist regarding said patient.

13. A system for monitoring a patient receiving human growth hormone (hGH) as an anti-aging therapy comprising
a specialist system accessible to a specialist monitoring said patient in communication with a non-specialist system accessible to a non-specialist administering said hGH,
a memory for storing data, and
a plurality of systems programs stored in said memory and selected from the group consisting of a screening program, a monitoring program, a dose calculation program, a calculated dose verification program, an administered dose verification program, a patient data program, an accessory function program, and combinations thereof.

14. The system of claim 13 wherein said monitoring program monitors the concentration of an analyte selected from the group consisting of insulin-like growth factor 1, testosterone, a thyroid hormone, and combinations thereof.

15. The system of claim 14 wherein the accessory function program is selected from the group consisting of posing and responding to queries, alerting the specialist or non-specialist, prompting for additional information, and combinations thereof.

16. A computer program product comprising
a computer usable medium having computer readable code embodied therein for determining an individualized dosing regimen of hGH to be administered to a patient for anti-aging hGH replacement, the computer usable medium comprising
means for screening a patient to determine candidacy for hGH replacement, means for calculating an initial dose of hGH for administering hGH to said patient, means for verifying said initial dose of hGH before administering hGH to said patient, means for verifying said initial dose of hGH after administering hGH to said patient, means for monitoring said patient, means for evaluating data for said patient, means for providing an accessory function, and combinations thereof.

17. The program of claim 16 wherein said means for screening a patient to determine candidacy for hGH replacement comprises determining said patient's concentration of insulin growth factor-1 (IGF-1) and testosterone, and accepting said patient for hGH replacement if said concentration of IGF-1 is at least 5% below a normal IGF-1 concentration or if said concentration of testosterone is at least 10% below a normal testosterone concentration.

18. The program of claim 16 wherein said means for calculating an initial dose of hGH for administering to said patient comprises evaluating parameters selected from the group consisting of age, gender, insulin growth factor 1 level (IFG-1), testosterone level, hematology profile results, chemistry profile results, and combinations thereof.

19. The program of claim 18 wherein said means for calculating said initial dose of hGH uses an input parameter of age ≧40 years, IGF-1 level ≧5% below normal, a testosterone level ≧10% below normal for males and ≧30% below normal for females, a hematology panel substantially within normal limits, and a chemistry panel substantially within normal limits.

20. The program of claim 16 wherein said means for verifying said initial dose of hGH before administering hGH to said patient comprises entering and transmitting said calculated dose to a specialist in hGH replacement therapy and receiving confirmation of said dose by said specialist before administering said dose to said patient.

21. The program of claim 16 wherein said means for verifying said initial dose of hGH after administering hGH to said patient comprises entering and transmitting said administered dose by a non-specialist in hGH replacement therapy to a specialist in hGH replacement therapy.

22. The program of claim 16 wherein said entering said administered dose comprises scanning an encoded dose contained on a vial containing hGH using a scanning apparatus to enter said encoded dose into said program.

23. The program of claim 16 wherein said means for monitoring said patient comprises monitoring said concentration of IGF-1 in said patient throughout said hGH dosing regimen.

24. The program of claim 23 wherein said monitoring comprises monitoring a parameter selected from the group consisting of IGF-1, IGF-1 binding protein, and testosterone in a graphical form.

25. The program of claim 16 wherein said monitoring further comprises incorporating an alert feature in said program for a pre-determined IGF-1 concentration.

26. The program of claim 16 wherein said means for evaluating data for said patient comprises evaluating objective and subjective criteria for said patient throughout and upon completion of said hGH dosing regimen.

27. A computer program product for use with a computer system, the computer program product comprising a computer usable medium having program code embodied in the medium for causing the computer system to establish an individualized dosing regimen of hGH replacement therapy comprising at least a screening program to determine candidacy of a patient for said therapy and a dose calculation program to determine an hGH dose for said patient.

28. The program of claim 27 further comprising a program selected from the group consisting of a dose verification program, a patient data program, a monitoring program, an accessory function program, and combinations thereof.
